(19) United States
(12) Patent Application Publication
(10) Pub. No.: US 2026/0361591 A1
(43) Pub. Date: Nov. 12, 2026

(54) Method and System to Facilitate Call-Control Continuity upon Loss of Connection

(71) Applicant: Stryker Corporation, Portage, MI (US)

(72) Inventors: Chris Gerardot, Portage, MI (US); David Stocking, Portage, MI (US)

(21) Appl. No.: 19/629,764

(22) Filed: Mar. 26, 2026

Related U.S. Application Data
(60) Provisional application No. 63/800,784, filed on May 6, 2025.

(51) Int. Cl. H04L 41/0246; G16H 40/67; H04L 65/10; H04L 65/1101; H04M 3/42

(52) U.S. Cl. CPC H04L 41/0246; G16H 40/67; H04L 65/1013; H04L 65/1101; H04M 3/42136

(57) ABSTRACT

A method and system to facilitate survival of call-control for a call between at least two devices located at the same facility as each other, where control of the call is handled by one or more nodes of a cloud-based platform through a network connection between the facility and the cloud-based platform, and where state data as to the call is maintained in the cloud-based platform. An example method includes (i) maintaining ongoing synchronization of the state data between the cloud-based platform and the facility, (ii) detecting a loss of connection between the facility and the cloud-based platform, and (iii) responsive to detecting the loss of connection, automatically causing one or more nodes at the facility to continue control of the call based on the synchronized state data, the one or more nodes at the facility corresponding with the one or more nodes of the cloud-based platform.

### REFERENCE TO RELATED APPLICATION

This application claims priority to U.S. Provisional Patent Application No. 63/800,784, filed May 6, 2025, the entirety of which is hereby incorporated by reference.

### BACKGROUND

Effective communication is often crucial within and among workplaces and other organizations. This is especially the case with healthcare facilities for example, where effective communication between healthcare workers such as physicians, nurses, and others helps to ensure patient safety, facilitate clinical decision-making, and enhance overall care quality. These communications may take the form of real-time call sessions, such as voice calls or video calls, among other possibilities.

In typical practice, when a calling user operating a calling device places a call to a called user operating a called device, the calling device may transmit to a call server a call-origination message that identifies the calling device and the called device. The call server may then responsively engage in further call-setup signaling such as (i) signaling with the called device to cause the called device to notify the called user of the incoming call (e.g., to ring), (ii) signaling with the calling device to notify the calling user that call setup is underway (e.g., to provide a ringback tone), and (iii) responding to the called user answering the call by engaging in further signaling with the calling device to set up an originating call leg, engaging in further signaling with the called device to set up a terminating call leg, and bridging the originating and terminating call legs together to enable the calling user and called user to communicate with each other. Alternatively, a call server may set up a more direct call connection between the devices or through a separate media bridge or server.

Example call-setup processing may also involve interaction with a number of other servers or resources. For example, when a call server receives a call-origination request from a calling device, the call server may responsively query a profile or account register to authenticate the caller and authorize the call and to acquire profile data indicating how to handle the call. Further, the call server may query an address-mapping database and a routing database to determine how to signal to the called device, among other possibilities.

### SUMMARY

Some facilities make use of on-premises call-control systems, such as a private branch exchange (PBX) and other systems comprising one or more servers physically present at the facilities. An on-premises call-control system at a given facility may be configured to handle setup and connections of calls between devices operating at the facility. Further, the on-premises system may provide connectivity with a public call-control system, such as the public switched telephone network (PSTN) or an internet-based call server, through which to connect calls between the facility and external users.

Unfortunately, however, implementing an on-premises call-control system may require substantial upfront investment in hardware, software, and infrastructure. Further, an on-premises call-control system may have limited scalability, may require regular maintenance and troubleshooting, and may become outdated without regular upgrades.

A distributed, cloud-based call-control system can help to address these and other issues, by hosting services in a distributed cloud infrastructure that allows call services to be centrally managed and deployed across potentially many facilities.

Among other possible arrangements, such a system may implement an actor model for distributed computing to provide a robust framework for distributed call control by enabling asynchronous, message-driven communication between isolated computational units, i.e., actors, while maintaining encapsulation and scalability. Alternatively, the system may implement a serverless model (e.g., with lambda nodes), which may usefully be scalable to millions of users.

With the actor model, by way of example, each actor may maintain exclusive control over its internal operation and may process messages sequentially. Multiple actors in a distributed cloud-based call-control system may thus interact with each other to accomplish call-control tasks such as setup and connection of calls.

In an example cloud-based call-control system, communication devices in a facility may be configured to interact with a cloud-based director actor that may coordinate call setup and other call control. In particular, each device at the facility may be configured to communicate with a cloud-based director actor through a cloud-based signaling-ingress node, and the cloud-based director actor may be configured to interact with a number of other actors, to facilitate call setup and other call control. Other arrangements may be possible as well.

Such a cloud-based call-control system may support setup and management of calls with external parties as well as calls between on-premises devices. For instance, when a device at a given facility seeks to call another device whether at the same facility or elsewhere, the calling device may engage in signaling, through an ingress node, with a cloud-based director actor, and the cloud-based director actor may responsively coordinate setup and connection of the call. For a call between an on-premises device and an remote, external device, the cloud-based director actor may set up a call connection that extends between the facility and the remote device. Whereas, for a call between devices at the facility, the cloud-based director actor may set up a call connection that extends within the facility between the devices.

This cloud-based call-control service assumes the existence of a robust network connection between the facility and the cloud-based director actor, and possibly with other cloud-based services or other resources. In typical practice, the facility would have a physical connection with the cloud. For instance, the facility may have an established internet connection and may include a router that enables devices at the facility to engage in internet, i.e., cloud, based communication.

As to calls between devices that are each physically or logically at the same facility as each other, each device may engage in interaction with the cloud-based call-control system, and the cloud-based call-control system may work to set up a call path within the facility between the devices. For instance, when a calling user operating a calling device in the facility places a call to a called user operating a called device in the facility, the calling device may engage in signaling, through the physical facility-cloud connection, with a cloud-based director actor. The cloud-based director actor may then responsively invoke services of various other cloud-based actors to carry out operations such as authentication, authorization, and route determination. And the cloud-based director actor may engage in further signaling, through the facility-cloud network connection, with the calling device and the called device to set up an in-facility call connection between the devices.

Unfortunately, a technological problem that may arise in this arrangement is that loss of network connectivity between the facility and the cloud could prevent or cause failure of in-facility call setup. This can be especially troubling when call setup between devices at the facility is underway. For instance, if called device at the facility has initially signaled to a cloud-based director actor and if call-setup signaling is thus in process to set up a call between the calling device and a called device also at the facility, and if the network connection between the facility and the cloud gets dropped, then setup of the call that is underway may fail. This may result in poor user experience and other issues, especially if the call placement is crucial.

The present disclosure provides a mechanism that may help to address this and other issues. In accordance with a disclosed example, when a cloud-based call-control system is processing setup of a call between devices at a facility, state data regarding the call setup will be regularly synchronized between the cloud and the facility. In the event the facility loses its connection with the cloud during the call-setup process, the call-setup process could then continue within the facility based on the synchronized state data, which may allow successful setup of the call between the devices.

Accordingly, in one respect, disclosed is a method to facilitate real-time survival of call-control for a call between at least two devices located at the same facility as each other, where control of the call is handled by one or more nodes of a cloud-based platform through a network connection between the facility and the cloud-based platform, and where state data as to the call is maintained in the cloud-based platform. The method involves maintaining ongoing synchronization of the state data between the cloud-based platform and the facility. Further, the method involves detecting a loss of connection between the facility and the cloud-based platform. And the method involves, responsive to detecting the loss of connection, automatically causing one or more nodes at the facility to continue control of the call based on the synchronized state data, the one or more nodes at the facility corresponding with the one or more nodes of the cloud-based platform.

Phrased another way, disclosed is a method to facilitate real-time call-control survivability for a call between at least two devices located at the same facility as each other, where control of the call is normally handled by one or more cloud-based applications through a network connection between the facility and the one or more cloud-based applications, where state data as to the call is maintained in one or more cloud-based storage servers. The method involves maintaining ongoing synchronization of the state data between (i) the one or more cloud-based storage servers and (ii) one or more local storage servers at the facility. Further, the method involves detecting a loss of connection between the facility and the one or more cloud-based applications. And the method involves, responsive to detecting the loss of connection, automatically causing one or more local applications at the facility to continue control of the call based on the synchronized state data in the one or more local storage servers, with the one or more local applications corresponding with the one or more cloud-based applications.

In yet another respect, disclosed is a computing system including at least one processor, non-transitory data storage, and program instructions stored in the non-transitory data storage and executable by the at least one processor to cause a computing system to carry out or facilitate operations such as those of the above methods for instance.

In still another respect, disclosed is non-transitory computer-readable data storage, having stored thereon program instructions executable by at least one processor of a computing system to carry out or facilitate operations such as those of the above methods for instance.

These, as well as other embodiments, aspects, advantages, and alternatives, will become apparent to those of ordinary skill in the art by reading the following detailed description, with reference where appropriate to the accompanying drawings. Further, it should be understood that the disclosure provided in this summary and elsewhere in this document is provided by way of example only and that numerous variations and other examples may be possible as well.

### BRIEF DESCRIPTION OF THE DRAWINGS

FIG. 1 is a simplified block diagram of an example system in which various disclosed features can be implemented.

FIG. 2 is another simplified block diagram of an example system in which various disclosed features can be implemented.

FIG. 3 is a simplified block diagram of an example device.

FIG. 4 is a flow chart illustrating an example method.

### DETAILED DESCRIPTION

The present disclosure will discuss example implementation in the context of a healthcare facility such as a hospital. It will be understood, however, that the disclosed principles could apply in numerous other contexts as well.

More generally, it will be understood that arrangements and processes disclosed herein could take various other forms. For instance, elements and operations could be re-ordered, distributed, replicated, combined, omitted, added, or otherwise modified. Further, elements described as functional entities could be implemented as discrete or distributed components or in conjunction with other components/modules, and in any suitable combination and location. Still further, various operations described as being carried out by one or more entities could be implemented by and/or on behalf of those entities, through hardware, firmware, and/or software, such as by one or more processing units executing program instructions stored in memory, among other possibilities.

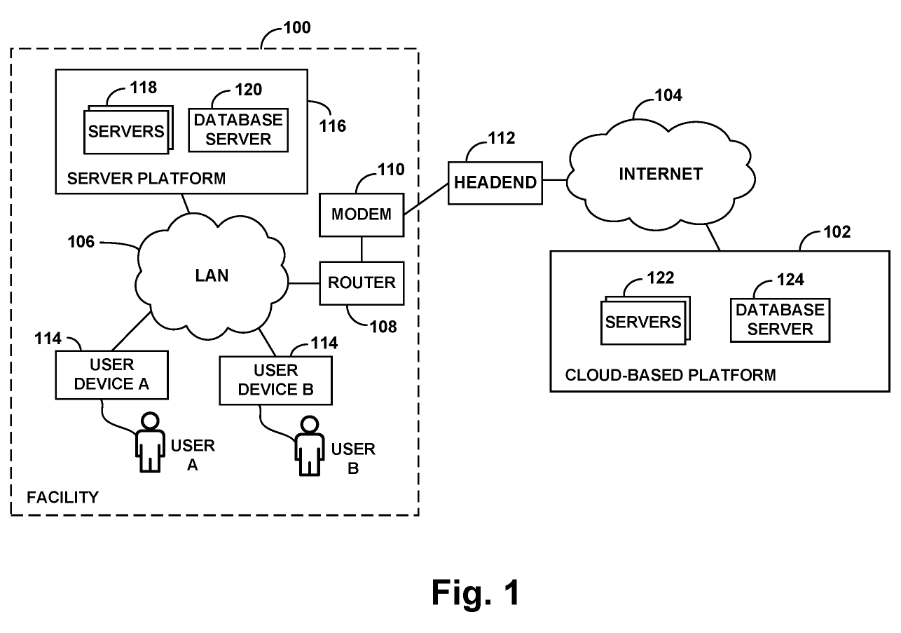

Referring to the drawings, as noted above, FIG. 1 is a simplified block diagram of an example system in which various disclosed features can be implemented. The example system includes a healthcare facility 100 and a cloud-based platform 102 accessible through a wide area network such as the internet 104.

The healthcare facility 100, which may comprise a hospital building or campus, is shown including multiple devices sitting as nodes on a local area network (LAN) 106, with the LAN 106 providing connectivity with the internet 104 to facilitate communication between the facility 100 and the cloud-based platform 102. As shown, the LAN 106 includes a router 108 configured to manage device connections and routing of data, and a modem 110 configured to connect with a headend 112 that provides internet connectivity.

Each device sitting as a node on the LAN 106 may have a network interface (e.g., a Wi-Fi interface or wired Ethernet interface) that may enable the device to engage in packet-switched communication on the LAN 106 and in turn on the internet 104. Further, each device may have a processor, non-transitory data storage, and program instructions stored in the non-transitory data storage and executable by the processor to carry out various device operations.

Shown among the devices on the LAN 106 at the facility 100 are two example end-user communication devices 114, user-device A and user-device B, operated by example users A and B. These communication devices could take any of various forms, configured to facilitate real-time call communication by and between the users. Without limitation, these user devices 114 could be caregiver communications devices such as badges (e.g., dedicated, hands-free communication badges worn and used by hospital staff), and/or the devices could be smartphones, watches, tablets, laptops, desktops, and/or other devices running communication applications.

Further shown on the LAN 106 at the facility 100 is a server platform 116 including one or more computer servers 118 that may host instantiated computing instances and other computing resources. For example, in accordance with the actor model for concurrent computation, the one or more servers 118 may host instantiated actors as fundamental units of computation, which may operate to support signaling and communication by and among the user devices, among other possibilities. Each such actor may or may not maintain its own individual state and may work asynchronously, processing messages and other signaling upon receipt, and providing associated output to facilitate interaction with others. Further, the server platform 116 is shown including a database server 120, defining a local database for the facility 100, which may also be an instantiated actor and/or may be external storage, among other possibilities.

The cloud-based platform 102 is likewise shown including one or more servers 122, which may similarly host instantiated computing instances and other computing resources. For instance, the one or more servers 122 may similarly host instantiated actors as fundamental units of communication, which may also operate to support signaling and communication by and among the user devices, among other possibilities. Further, the cloud-based platform 102 is similarly shown including a database server 124, defining a cloud database, which may also be an instantiated actor and/or may be external storage, among other possibilities.

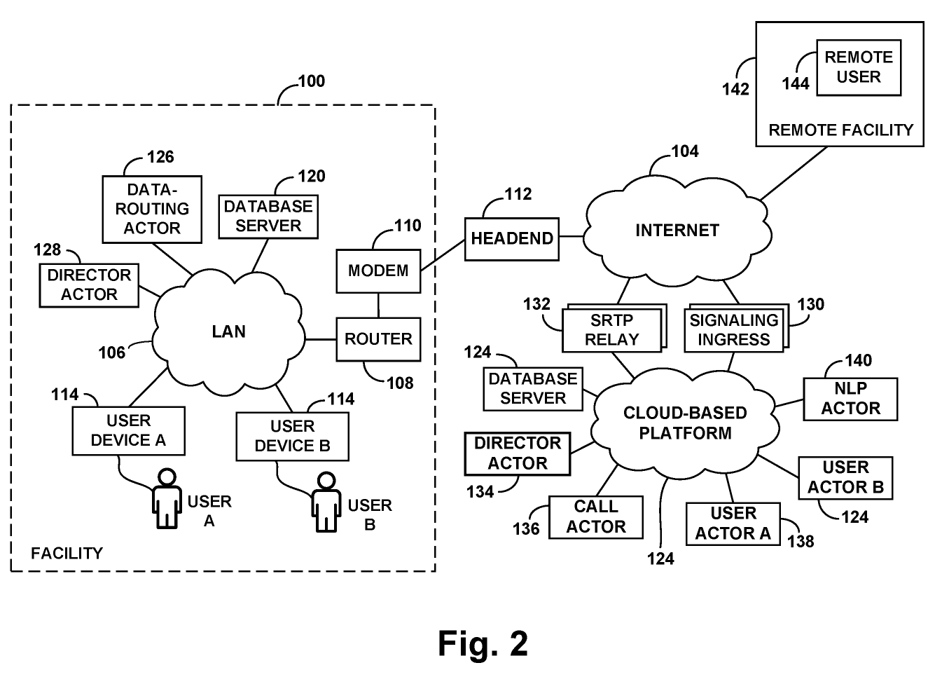

FIG. 2 illustrates a more specific configuration of the arrangement of FIG. 1, as an example arrangement in which the cloud-based platform 102 supports real-time packet-based call communication (e.g., voice or video calling) by and between the example user devices 114. In practice, an operator of the cloud-based platform 102 may provide this service, and an operator of the facility 100 may subscribe to the service.

FIG. 2 shows, at the facility 100, the user devices A and B and the local database 120, as well as a local data-routing actor 126, and a local director actor 128. With this arrangement, the local data-routing actor 126 helps to control routing of communications to and from various entities such as the various actors on the LAN 106. And the local director actor 128 helps to coordinate and manage various operations such as spawning of new local actors, among other possibilities.

The figure then shows in the cloud-based platform 102, accessible via the internet 104, a signaling ingress 130 defining a node with which entities the facility 100 can communicate to facilitate their interaction with various other cloud-based entities. Further, the figure shows on the internet a secure real-time transport protocol (SRTP) relay 132 with which entities at the facility 100 may be able to engage in real-time packet-based communication such as exchange of voice and/or video communication for instance.

In addition, the figure shows in the cloud-based platform the cloud database 124 as well as a number of actors or other resources, including a director actor 134, a call actor 136, user actors 138, and a natural language processing (NLP) actor 140. With this arrangement, the director actor 134 may operate to route communications to and from the other cloud-based entities such as the signaling ingress 130, the cloud database 122, and the various actors, among other possibilities. The call actor 136 may operate to control call setup and to manage ongoing calls. The user actors 138 may represent the respective physical user devices 114, and may therefore include a user-actor A representing user-device A, and a user-actor B representing user-device B. Further, the NLP actor 140 may operate to carry out NLP processing, such as to interpret voice audio provided by users and to provide associated text, voice, or other data output to facilitate associated operations.

The figure also illustrates a remote facility 142, where a remote user 144 may operate. In practice, the cloud-based platform 124 may usefully support inter-facility communications, such as communications between a user at facility 100 and the remote user 144 at the remote facility 142. Further, the cloud-based platform 124 may also usefully support intra-facility communications, such as communications internally between users at facility 100.

In practice, each user device at facility 100 may register with or to facilitate establishment of a respective user actor 138. Each user device may then engage in signaling with its respective user actor 138, and its respective user actor 138 may in turn interact with other actors to facilitate associated processing.

With this arrangement, in an example implementation, user A may place a call to user B through voice interaction. For instance, user A may speak such as “Call user B” into device A. In response, device A may responsively signal to user-actor A, through local-data routing actor 126, signaling ingress 130, and director actor 134. User-actor A may then responsively signal to the NLP actor 140 to trigger NLP processing, and device A may transmit the user’s voice audio through the SRTP relay 132 to the NLP actor 140. The NLP actor 140 may then responsively translate the user’s voice command into a call-initiation command and may transmit the call-initiation command to user-actor A. And user-actor A may then responsively transmit to the call actor 136 a call-initiation request message, requesting initiation of a call from user device A to user device B.

On receipt of this call-initiation request message, the call actor 136 may then send a call-invitation message to user-actor B, which may forward the call-invitation to user-device B, triggering ringing or other incoming-call notification at device B. When user B answers this call or device B automatically answers this call, device B may send an acceptance message to user-actor B, which may forward the acceptance to the call actor 136. The call actor 136 may then spawn a media-router actor such as another SRTP instance, that may operate to bridge the requested call. This media-router actor may then allocate ports and may forward a media-configuration message respectively, possibly via the call actor 136, to each of user-actors A and B. And user-actors A and B may forward associated connection requests to their respective devices A and B, to establish for each device a respective connection with the media-router actor. And the media-router actor may then bridge those connections to enable the devices and thus the users to communicate with each other.

In this process, each actor may maintain its own state records as noted above, and may update those state records as its operational state changes over time. For instance, when a user actor sends or receives a call-invitation message, the user actor may update its internal state records to indicate its sending or receipt of that call-invitation message. Likewise, when a user actor sends or receives a response to such a call-invitation message, the user actor may update its internal state records to indicate its response. Similarly, when the call actor 136 sends or receives a call-invitation message or response, the call actor 136 may update its internal state records accordingly. This same internal state updating may occur for any of various entities involved with the call setup process.

As noted above, a technical problem that can arise in practice is that the facility 100 may lose its internet connection while this call setup process is underway. If that happens, setup of the call from device A to device B will fail. Further, if the facility 100 has lost its internet connection even when call setup is not underway, devices at the facility 100 will lose their ability to set up calls with each other.

As further noted above, a technical solution to this problem is to dynamically synchronize operational-state records between the cloud-based platform 102 and the facility 100, and to automatically transition from call-control processing in the cloud-based platform 102 to call-control processing in the facility 100 when the facility loses its cloud connectivity. The state data at issue here could include data regarding the state of a call, such as call-session state and/or call-participant state (e.g., user state), among other possibilities.

Dynamically synchronizing operational state records between the cloud-based platform 102 and the facility may involve maintaining in the local database 120 at the facility 100 a copy or other representation of the state of various entities of the cloud-based platform 102. Various data-management constructs could be used to facilitate this. For instance, whenever the state of any entity in the cloud-based platform 102 changes, that entity may publish an indication of the state change, and that published state change may propagate to the local database 120. By way of example, when an entity such as a user actor 138 or the call actor 136 sends or receives a call-setup-related message such as one of those noted above and updates its call-setup state accordingly, the entity may publish an indication of its call-setup state change, and the entity’s new call-setup state may get recorded in the local database 120.

Various data-distribution constructs could be used to facilitate this synchronization of state records. By way of example, the involved entities could apply a RAFT consensus algorithm in the context of a distributed database. RAFT can operate in a known manner to distribute state data across a cluster of computing systems so as to help ensure that the nodes in the cluster agree upon the same series of state transitions as each other. Applying RAFT in the present context, when a node of the cloud-based platform 102 experiences a state change, the node propagates the state change through a leader-driven log replication process, with a leader node writing the state change in a persistent log and sending the log entry to each follower node, and with each follower node updating their state records accordingly. With this process, the local database 120 could be a follower node in this RAFT process and could therefore maintain a record of the current state of each of the various entities of the cloud-based platform.

A RAFT arrangement may include a single leader node and multiple follower nodes. In a variation of that arrangement for present purposes, the cloud-based platform 102 and the facility 100 may each have a respective RAFT leader node and a number of RAFT follower nodes. With this variation, the leader nodes of the cloud-based platform 102 and of the facility 100 may each have a persistent log, synchronized with each other to facilitate the present operations with respect to their respective follower nodes.

Other constructs could be used as well. For instance, with a serverless model and/or in other arrangements, the entities could make use of a distributed hash table (DHT), among other possibilities.

To detect when the facility 100 loses connectivity with the cloud-based platform 102 so as to trigger transitioning to call-control processing in the facility 100, local data-routing actor 126 may monitor the connection with the cloud-based platform. For instance, the local data-routing actor 126 may engage in regular heartbeat signaling with the signaling ingress 130, the director actor 134, and/or one or more other entities of the cloud-based platform 102. Loss of that heartbeat signaling (e.g., absence of signaling from the cloud-based platform) may represent loss of connectivity between the facility 100 and the cloud-based platform 102.

In response to the local data-routing actor 126 detecting loss of connectivity with the cloud-based platform, the local data-routing actor 126 may responsively signal to the local director actor 128 to trigger associated processing. Local director actor 128 may then responsively engage in processing to instantiate local versions of the various cloud-based actors and to provision those local actors with the most recent synchronized state information. For instance, the local director actor 128 may read state data from the local database 120 and may launch new local instances of a signaling ingress, an SRTP relay, user actors, and call actor, each with restored state data that was most recently synchronized with the facility 100. More particularly, with an example actor model, instantiating a new actor node may comply with an actor framework and may involve (i) registering the new actor node with the actor system, (ii) passing configuration data to the actor node, and (iii) establishing an address or path to facilitate messaging with other actor nodes in the system. Further, the local director actor 128 may signal to impacted local entities, such as to devices A and B, to notify them that they should proceed to communicate through the local signaling ingress with the local entities rather than through the cloud-based signaling ingress 130 with the cloud-based entities.

This process may facilitate real-time survival of call-control as to call setup that is underway, if the facility 100 loses its connection with the cloud-based platform 102.

For instance, consider a scenario with the above-noted call placement process, where user-actor A in the cloud-based platform 102 has sent a call-origination message to the call actor 136 in the cloud-based platform and the call actor 136, and the call actor 136 has responsively sent a call-initiation message to user-actor B in the cloud-based platform 102. And assume that, at that point, the facility 100 loses connectivity with the cloud-based platform 102. In response to that loss of connectivity, the local director actor 128 may launch and provision the actors as noted above. Based on the synchronized state data, the local instance of user actor B may then continue with the call setup process, by signaling within the facility to user device B and, upon receipt of a response from user device B then signaling with the local instance of the call actor, and so forth. Ultimately through this process, the local instance of the call actor may thus set up the call locally between user device A and user device B, allowing users A and B to communicate with each other.

Synchronizing of the state data between the cloud-based platform 102 and the facility 100 may also facilitate starting of call placement in a scenario where the facility 100 has lost connectivity with the cloud-based platform 102. For instance, after the local data routing actor 126 detects the loss of connectivity and responsively causes the local director 128 to launch local instances of the various actors, if device A then seeks to place a call to device B, they would interact with the local instances of their respective user actors, which would interact with the local instances of the other actors to facilitate setting up the call.

Further, synchronizing of the state data between the cloud-based platform 102 and the facility 100 may also facilitate management of an ongoing call that is already in progress between devices A and B. In that case, when the facility 100 loses connection with the cloud-based platform 102 during the call, the newly instantiated local instances of the various actors can continue management of the call, and the call may also handover from a cloud-based SRTP relay to a local instance of an SRTP relay. Examples of state as to an ongoing call may include mute state, volume state, call-participant data, among others. With this transition, call-control operations such as user-requested muting, volume changes, adding or removing of participants, or terminating of the call, among other possibilities, could be managed by the local actor instances.

In an alternative implementation, some or all of the actors may be instantiated locally at the facility 100 in parallel with their being instantiated in the cloud-based platform 102. Further, those local instances may maintain state synchronization with their cloud-based counterparts using RAFT, DHT, or another process. When the facility 100 loses its connectivity with the cloud-based platform 102, it may then be a simple manner of the local director actor 128 notifying the local actor instances and the local user devices to facilitate call-control processing within the facility 100.

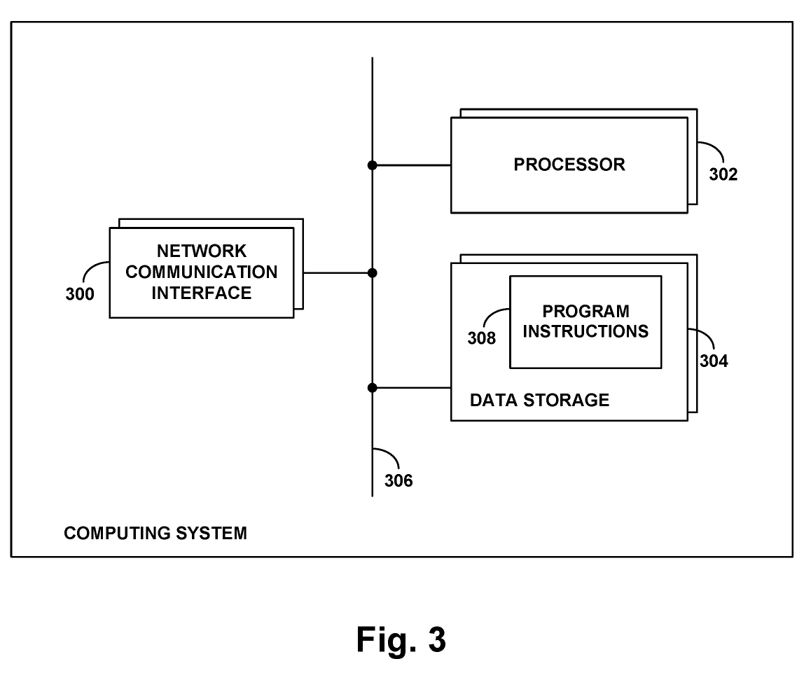

FIG. 3 is a simplified block diagram illustrating components of an example device, which could be any of the entities discussed above, among other possibilities. As shown in FIG. 3, the device includes a network communication interface 300, a processor 302, and non-transitory data storage 304. These components can be integrated together and/or communicatively linked together in various ways. For instance, the components can be linked together through a system bus, network, or other connection mechanism 306 and/or can be integrated together in various ways.

The network communication interface 300 can include components that enable the device to engage in network communication with other devices. The network communication interface 300 may facilitate wireless and/or wired communication and take associated forms, including or interworking with associated drivers.

The processor 302 may comprise one or more general purpose processors (e.g., microprocessors) and/or one or more specialized processors (e.g., digital signal processors (DSPs), graphics processing units (GPUs), neural processing units (NPUs), etc.) The non-transitory data storage 304 may then comprise one or more volatile and/or non-volatile storage components (e.g., flash, optical, magnetic, read only memory (ROM), random access memory (RAM) (e.g., dynamic RAM (DRAM), static RAM (SRAM), or double data rate RAM (DDRAM)), electronically programmable read only memory (EPROM), and/or electronically erasable programmable read only memory (EEPROM), etc.), and may be integrated in whole or in part with the processor 302 or may be provided separately.

As further shown, the data storage 304 may store program instructions, which may be executable by the processor 302 to carry out various operations discussed herein. For instance, a device that hosts an actor node may include program instructions defining operation of that actor node and may further maintain state data representing the internal state of that actor node.

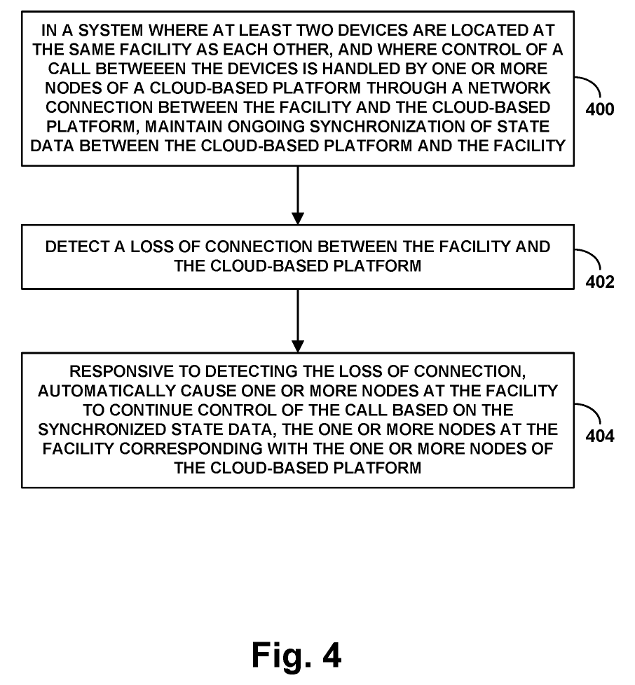

FIG. 4 is a flow chart illustrating an example method that could be carried out in accordance with the present disclosure, to facilitate real-time survival of call-control for a call between at least two devices located at the same facility as each other, wherein control of the call is handled by one or more nodes of a cloud-based platform through a network connection between the facility and the cloud-based platform, and wherein state data as to the call is maintained in the cloud-based platform. As shown in FIG. 4, at block 400, the example method involves maintaining ongoing synchronization of the state data between the cloud-based platform and the facility. Further, at block 402, the method involves detecting a loss of connection between the facility and the cloud-based platform. And at block 404, the method involves, responsive to detecting the loss of connection, automatically causing one or more nodes at the facility to continue control of the call based on the synchronized state data, wherein the one or more nodes at the facility correspond with the one or more nodes of the cloud-based platform.

In line with the discussion above, the nodes of the cloud-based platform could comprise actor nodes of a computational actor model or could take other forms. Further, the act of automatically causing the one or more nodes at the facility to continue control of the call based on the synchronized state data could involve instantiating at least one of the one or more nodes at the facility, which may include registering the node with a framework system, passing state or other configuration data to the node, and establishing an address of the node in the system.

As further discussed above, the act of maintaining the ongoing synchronization of the state data could involve using a RAFT consensus algorithm to maintain the ongoing synchronization.

In addition, as discussed above, the facility at issue could be a healthcare facility, and the call could be a call between caregiver communication devices in the healthcare facility. For instance, the call could be a call between badges in the facility, among other possibilities. Further, as noted above, the call could take the form of a voice call (e.g., a call without video) or a video call (e.g., a call with video and voice).

As additionally discussed above, the state data at issue in this method could take various forms. For instance, the state data could include call-setup state data defining state of processing to initially set up the call. Further, the state data could include ongoing call state data. Yet further, the state data could comprise user state, such as state of a user device such as that the user device is engaged in call setup or is engaged in a call, among other possibilities.

The present disclosure also contemplates non-transitory data storage (e.g., one or more non-transitory computer-readable medium components (e.g., flash, optical, magnetic, ROM, RAM) (e.g., DRAM, SRAM, or DDRAM), EPROM, and/or EEPROM, and/or other computer-readable media, etc.)) holding program instructions executable by at least one processor (e.g., of one or more devices) to carry out various operations described herein, such as those of the example method for instance.

Further, the present disclosure contemplates a system, comprising at least one processor, non-transitory data storage, and program instructions stored in the non-transitory data storage and executable by the at least one processor to carry out various operations described herein, such as those of the example methods for instance.

Further, the present disclosure contemplates a computer program comprising a set of program instructions executable by at least one processor of a computing system to carry out (e.g., to cause the computing system to carry out) various operations described herein, such as those of the example methods for instance. In an example implementation, the computer program could further be stored in non-transitory data storage such as that noted above, among other possibilities.

Exemplary embodiments have been described above. Those skilled in the art will understand, however, that changes and modifications may be made to these embodiments without departing from the true scope and spirit of the invention.

What is claimed is:

1. A method to facilitate survival of call-control for a call between at least two devices located at a same facility as each other, wherein control of the call is handled by one or more nodes of a cloud-based platform through a network connection between the facility and the cloud-based platform, and wherein state data as to the call is maintained in the cloud-based platform, the method comprising:
maintaining ongoing synchronization of the state data between the cloud-based platform and the facility;
detecting a loss of connection between the facility and the cloud-based platform; and
responsive to detecting the loss of connection, automatically causing one or more nodes at the facility to continue control of the call based on the synchronized state data, wherein the one or more nodes at the facility correspond with the one or more nodes of the cloud-based platform.

2. The method of claim 1, wherein the nodes of the cloud-based platform comprise actor nodes of a computational actor model.

3. The method of claim 1, wherein automatically causing the one or more nodes at the facility to continue control of the call based on the synchronized state data comprises instantiating at least one of the one or more nodes at the facility.

4. The method of claim 1, wherein maintaining the ongoing synchronization of the state data comprises using a RAFT consensus algorithm to maintain the ongoing synchronization.

5. The method of claim 1, wherein the facility is a healthcare facility, and wherein the call is a call between caregiver communication devices in the healthcare facility.

6. The method of claim 5, wherein each caregiver communication device comprises a badge.

7. The method of claim 1, wherein the state data comprises call-setup state data defining state of processing to initially set up the call.

8. The method of claim 1, wherein the state data comprises ongoing call state data.

9. The method of claim 1, wherein the state data comprises user state.

10. The method of claim 1, wherein the call is a voice call.

11. A system comprising:
at least one processor;
non-transitory data storage; and
program instructions stored in the non-transitory data storage and executable by the at least one processor to carry out operations to facilitate survival of call-control for a call between at least two devices located at a same facility as each other, wherein control of the call is handled by one or more nodes of a cloud-based platform through a network connection between the facility and the cloud-based platform, and wherein state data as to the call is maintained in the cloud-based platform, the operations including:
maintaining ongoing synchronization of the state data between the cloud-based platform and the facility,
detecting a loss of connection between the facility and the cloud-based platform, and
responsive to detecting the loss of connection, automatically causing one or more nodes at the facility to continue control of the call based on the synchronized state data, wherein the one or more nodes at the facility correspond with the one or more nodes of the cloud-based platform.

12. The system of claim 11, wherein the nodes of the cloud-based platform comprise actor nodes of a computational actor model.

13. The system of claim 11, wherein maintaining the ongoing synchronization of the state data comprises using a RAFT consensus algorithm to maintain the ongoing synchronization.

14. The system of claim 11, wherein the facility is a healthcare facility, and wherein the call is a call between caregiver communication devices in the healthcare facility.

15. The system of claim 14, wherein each caregiver communication device comprises a badge.

16. The system of claim 11, wherein the state data comprises call-setup state data defining state of processing to initially set up the call.

17. The system of claim 11, wherein the state data comprises ongoing call state data.

18. The system of claim 11, wherein the state data comprises user state.

19. The system of claim 11, wherein the call is a voice call.

20. At least one non-transitory computer-readable medium having stored thereon program instructions executable by at least one processor to carry out operations to facilitate survival of call-control for a call between at least two devices located at a same facility as each other, wherein control of the call is handled by one or more nodes of a cloud-based platform through a network connection between the facility and the cloud-based platform, and wherein state data as to the call is maintained in the cloud-based platform, the operations comprising:
maintaining ongoing synchronization of the state data between the cloud-based platform and the facility;
detecting a loss of connection between the facility and the cloud-based platform; and
responsive to detecting the loss of connection, automatically causing one or more nodes at the facility to continue control of the call based on the synchronized state data, wherein the one or more nodes at the facility correspond with the one or more nodes of the cloud-based platform.
